(19) United States
(12) Patent Application Publication
(10) Pub. No.: US 2019/0059861 A1
(43) Pub. Date: Feb. 28, 2019

(54) ADVANCED SPECIFIC HARVEST, CONTAINMENT, AND DEPLOYMENT SYSTEM (ASHCADS)

(71) Applicant: PolarityTE, Inc., Salt Lake City, UT (US)

(72) Inventors: Denver Lough, Park City, UT (US); Edward Swanson, Salt Lake City, UT (US)

(73) Assignee: PolarityTE, Inc., Salt Lake City, UT (US)

(21) Appl. No.: 16/109,341

(22) Filed: Aug. 22, 2018

Related U.S. Application Data
(60) Provisional application No. 62/548,869, filed on Aug. 22, 2017.

(51) Int. Cl. A61B 10/00; B01L 3/00; A61K 31/16; A61K 31/137; A61K 31/7036; A61B 10/02; A61B 10/06; A61B 50/30; A61F 17/00; A61M 19/00; A61B 42/40

(52) U.S. Cl. CPC A61B 10/0096; B01L 3/50; A61K 31/16; A61K 31/137; A61K 31/7036; A61B 10/0266; A61B 2050/0014; A61B 50/3001; A61F 17/00; A61M 19/00; A61B 42/40; B01L 2200/141; B01L 2200/185; A61B 10/06

(57) ABSTRACT

Kits and methods are provided for harvesting samples such as cells or tissue including skin tissue for use in research and therapeutic protocols. Kits and methods for deploying grafts, constructs or other products for therapeutic use are also provided. Use of the kits temporarily preserves the viability and/or function of the sample (such as a tissue sample) and/or therapeutic agent.

### CROSS REFERENCE TO RELATED APPLICATIONS

This application claims the benefit of U.S. provisional application No. 62/548,869, filed Aug. 22, 2017, which is incorporated herein in its entirety by reference.

### FIELD OF THE INVENTION

The present invention relates generally to a kit for harvesting a sample (e.g. cells, organic and inorganic articles) from a target (including for instance a mammalian subject) and methods of using the same. The present invention also extends to a kit for deploying an agent (such as a therapeutic agent formed from a tissue sample such as a graft, construct, or product) prepared from the harvested sample to an end user and methods of using the same. By using the kits, the viability and/or function of the sample and/or agent is at least temporarily preserved in a condition substantially pristine to the harvested condition of the sample.

### BACKGROUND OF THE INVENTION

Successful use of harvested samples may require specific handling and transportation requirements to maintain the viability and/or function of the sample. For instance, transplantation of cells or tissues is useful to treat certain diseases, disorders, and injuries. But success in transplanting cells or tissues into a recipient depends on the quality of the cells or tissues used for transplant. In turn, the quality of those cells or tissues depends on the methods used to harvest, store and/or transport the same. In order to maintain quality, there is a need to control and monitor the environment of the harvested samples.

There remains a need for new approaches to promoting survivability and/or function of harvested specimens. There remains a further need for new approaches that are efficient and reduce risk of contamination of the specimen. There remains a further need for new approaches that allow for rapid turn-around from sample harvest to agent deployment across the globe that is reliable, traceable, and communicative. For instance, there is a need to deliver patient-specific cell or tissue, or cell- or tissue-derived therapy within 48 hours of sample harvest for optimal therapy performance and to fit within highly confined patient treatment schedules. Additionally, many therapies and research products that require short periods of time from sample harvest to agent deployment require strict environmental control and monitoring, real-time tracking, and capabilities to solve challenging logistical issues that arise from traditional delivery services.

More particularly, there remains a need for innovative approaches to facilitate and reduce barriers to the use of cell and/or tissue transplantation as a treatment alternative to off-the-shelf products through efficient and convenient methods of harvest, storage, transport, or deployment of the cells or tissues. With recent innovation in biomedical technology, improved outcomes may be achieved with patient-specific and disease-specific therapies, but unique solutions are required to integrate and promote adoption of these therapies into current medical practice.

### SUMMARY OF THE INVENTION

The kits disclosed herein can advantageously promote survivability and/or function of a harvested sample or agent created therefrom (e.g, therapeutic agent). The kits disclosed herein can also advantageously reduce risk of contamination of a harvested sample or agent created therefrom (e.g., therapeutic agent).

In aspects, the kits disclosed herein can advantageously facilitate patient- or disease-specific therapies (e.g., by conveniently providing and combining the various articles contained therein and decreasing the time otherwise spent by patients and medical care providers in obtaining, transporting, and deploying samples).

In aspects, the kits disclosed herein can advantageously facilitate harvesting and/or deployment in a remote and minimally equipped location (e.g. without an operating room). The kits disclosed herein can also advantageously facilitate patient- or disease-specific therapy creation at a remote location distant from the location of the patient.

In aspects, the kits disclosed herein can advantageously facilitate tracking, monitoring, and control of various aspects of the kit (e.g. geographic location, environmental factors, etc.).

In aspects, the kits can advantageously facilitate tumor specific therapies.

In aspects, the kits disclosed herein can advantageously facilitate agricultural and other plant-based hybridization, for example, where harvesting and/or deployment is performed in a remote and minimally equipped location (e.g. without a diverse and sophisticated plant nursery), and/or where plant and agricultural hybridization is performed at a remote location distant from the location of the planting.

In aspects, the kits disclosed herein can advantageously decrease the time spent by farmers in obtaining, transporting, and deploying hybridized and engineered plant samples.

In a first aspect, the present invention is a kit for harvesting a sample from a target. Advantageously, the kit permits a reduction in risk of contamination of the sample and/or increases the efficiency of obtaining, preserving or transporting the same. Certain components of the harvest kit are used to preserve and, optionally, to transport the sample to a second location for research, processing, or therapeutic purposes. Certain components of the harvest kit can be used to control and monitor the harvest sample environment to optimize and maintain sample integrity and function. In addition, other components of the kit can be used for geographically locating the sample and monitoring the physical state of the container and its contents to improve logistical coordination and assure transport quality upon arrival.

It shall be noted that the disclosed kit, by providing the noted elements in a combination, increases the efficiency of obtaining, storing, transporting, and deploying a sample, reduces degradation of the sample, and facilitates remote in-field use. For instance, in the case of a tissue sample, the kit facilitates harvest, storage, transportation and deployment by various types of medical practitioners (doctors, nurses, etc.) in sparsely equipped field locations outside of an operating room (e.g., clinic, bedside, emergency department, intensive care unit, forward operating center, etc.) without the need for extraneous equipment or supplies.

In a second aspect, the present invention is a kit for deploying an agent (such as a therapeutic agent formed from a tissue sample such as a graft, construct, or product). In exemplary embodiments, the deployment kit is transported to a second location for use such as therapeutic use via implantation of a therapeutic agent in a recipient. In embodiments, instead of the recipient being an end user or patient, the recipient can be a bioreactor (e.g., a mammal) for processes the sample prior to final implantation into a recipient for therapeutic use. In the case of such therapeutic use the recipient may be the same or different than the subject that supplied a cell, tissue, or fluid sample, i.e., the therapeutic agent may be autologous or allogeneic.

In an exemplary embodiment, a kit of the present invention comprises, alternatively or in combination: an insulated mailer or environmentally controlled shipment package, a temperature tracker, a cooling component (e.g., a freezer pack), a biohazard bag (e.g., a UN3373 compliant biohazard bag), a deployment device such as an irrigation syringe (e.g., 30 cc or 60 cc irrigation syringe), a local anesthetic (e.g., lidocaine), a vasoconstrictor (e.g., epinephrine), an absorbent pack, sterile gloves, a laceration tray, an antimicrobial scrub tray (e.g., a povidone iodine scrub tray), a sample collection tool such as a biopsy device (e.g., a scalpel), a skin marking device (e.g., a surgical marker), a label, a suture, and a dressing (e.g., silicone or ioban dressing).

In an exemplary embodiment, the kit comprises at least one: (i) insulated mailer or environmentally controlled shipment package; (ii) temperature tracker, (iii) cooling component, (iv) biohazard bag, (v) local anesthetic, (vi) vasoconstrictor, (vii) absorbent pack, (viii) sterile gloves, (ix) forceps, (x) scissors, (xi) needle driver, (xii) antimicrobial agent (e.g., povidone iodine or gentamicin), (xiii) biopsy device, (xiv) skin marking device, and (xv) suture.

In one embodiment, certain kit components are present in multiples, i.e., more than one of the component is provided. In a particular embodiment, the kit comprises more than one scalpel. In another particular embodiment, the kit comprises more than one biohazard bag (e.g., two or three biohazard bags). In another particular embodiment, the kit comprises more than one label. In a further particular embodiment, the kit comprises more than one dressing.

The kit can contain a box and/or an insulated mailer and/or an environmentally controlled shipment package and/or a temperature tracker and/or a cooling component and/or a biohazard bag and/or a sterile container and/or a deployment device such as an irrigation syringe and/or a local anesthetic and/or a vasoconstrictor and/or an absorbent pack and/or sterile gloves and/or forceps and/or scissors and/or a needle driver and/or an antimicrobial agent and/or a laceration tray and/or an antimicrobial scrub tray and/or a sample collection tool such as a biopsy device (e.g., a scalpel) and/or a skin marking device and/or a label and/or a suture and/or a dressing and/or a transport medium and/or a hemostatic agent and/or a means for remote monitoring and communication (e.g., real-time monitoring and communication such as a geographical locating device such as a GPS transponder chip) and/or an environmental monitoring device and/or antimicrobial monitoring and/or microbial control means and/or personal protective equipment and/or a physical security feature and/or a digital security feature and/or a traceability feature and/or a tamper prevention feature and/or a tamper evident feature and/or combinations of two or more of these components.

In one embodiment, the local anesthetic is lidocaine-epinephrine prepared as an injectable solution and provided in sterile vial with a sterile syringe and needle for injection. In a particular embodiment, the lidocaine is lidocaine hydrochloride. In another particular embodiment, the injectable solution is from 1% to 5% lidocaine and more particularly, 1% or 2% lidocaine. In a further particular embodiment, the ratio of epinephrine is 1:100,000. In a further particular embodiment, the ratio of epinephrine is 1:200,000. In another particular embodiment, the local anesthetic does not contain a vasoconstrictor.

In a further embodiment, whether with a vasoconstrictor or without a vasoconstrictor, the local anesthetic is XYLOCAINE®. In a particular embodiment the injectable solution is from 1% to 5% XYLOCAINE® and more particularly, 1% or 2% XYLOCAINE®. In a further particular embodiment, the local anesthetic prepared as an injectable, with or without the addition of vasoconstrictor is bupivacaine (e.g., MARCAINE®). In a particular embodiment, the bupivacaine is bupivacaine hydrochloride. In another particular embodiment, the injectable solution is from 0.25% to 0.75% bupivacaine.

Optionally, the kit may contain one or more additional components used in harvesting, preserving, transporting, or deploying tissue.

In one embodiment, the kit further comprises an effective amount of transport media. The transport medium may be any suitable transport medium. In a particular embodiment, the transport medium is a sterile medium with serum or sterile isotonic solution or sterile crystalloid solution (iso-, hyper-, or hypo tonic). Optionally, the transport medium may contain one or more antibiotics. In one embodiment, the antibiotic is gentamicin. In a further embodiment, the antibiotic is bacitracin.

In another embodiment, the kit further comprises a sterile container (e.g., a cup) for storing a tissue sample.

In a further embodiment, the kit further comprises forceps.

In a third aspect, the present invention is a method of using the disclosed kit to harvest a tissue sample from a subject. By using the kit, the viability and/or function of the biological sample is temporarily preserved.

The sample may be any suitable tissue. In one embodiment, the tissue is skin.

In an exemplary embodiment, the method comprises (i) cleaning the skin; (ii) injecting an effective amount of the local anesthetic to produce an insensate skin area; (iii) biopsying the skin at a target site within the insensate skin area using the biopsy device; (iv) excising the biopsy to provide a skin biopsy and a biopsy cavity; and (v) applying at least one dressing over the area of the biopsy cavity.

In one embodiment, the skin is cleaned using a betadine wet-prep system and the area to be biopsied is draped.

In one embodiment, the biopsy technique permits a full-thickness tissue sample. In a particular embodiment, the biopsy technique is punch biopsy. In another particular technique, the biopsy technique is an excisional technique. The particular method dictates the type of biopsy device.

The size of a tissue sample may vary. In one embodiment, the size of a tissue sample is from about 1 mm² to about 10 mm². In another embodiment, the size of a tissue sample is greater than about 10 mm².

In a fourth aspect, the present invention is a method of preserving and, optionally, transporting a tissue sample to a second location for research, processing, or therapeutic purposes using certain components of the disclosed kit.

In one embodiment, a tissue sample is transported in the sterile container (e.g., a cup) within at least one biohazard bag housed within an insulated mailer (e.g., an insulated box) or environmentally controlled shipment package. The sterile container and at least one biohazard bag, optionally in combination with the cooling device, preserves the viability and functionality of a tissue during transport. In a particular embodiment, the cooling device is a freezer pack, dry ice, wet ice, or liquid nitrogen. In one embodiment, the cooling device does not permit tissue to freeze.

In exemplary embodiments, a tissue sample is transported to a second location within 24 hours, 48 hours, 72 hours, or 96 hours.

In exemplary embodiments, a temperature tracker is included within the kit to monitor the temperature of a tissue sample. In exemplary embodiments, an environmentally controlled shipment package with environmental monitors and a temperature tracker is included within the kit. In a particular embodiment, a temperature tracker is an activated strip. In a particular embodiment, the temperature tracker is a real-time continuous monitor capable of transmitting data in real-time to anywhere in the world, including transmitting data to multiple locations and users in real-time simultaneously. The real-time continuous monitor is capable of transmitting and connecting to remote locations for real-time primary and backup data storage for real-time or future review.

In one embodiment, the method further comprises processing a tissue sample following transportation to the distant site to provide a therapeutic agent (e.g., graft, construct or product). In a particular embodiment, the graft or cell therapy is suitable for use in restoring skin damaged by disease or injury. In a particular embodiment, the graft or cell therapy is suitable for use in restoring bone damaged by disease or injury. In a particular embodiment, the therapeutic agent is an immunotherapy. In a particular embodiment, the therapeutic agent is a vaccine. For example, in a particular embodiment, the vaccine is created from appropriately and efficiently harvested, stored, and transported tissue from a patient (e.g. a tumor biopsy) using the harvest kit in order to deliver a patient-specific, tumor-specific vaccine at the second location, and then returned using the deployment kit to maintain stable environmental and handling conditions. In a particular embodiment, the therapeutic agent created at the second location is the processed serum collected from a human or animal that has known immunity against a disease or infectious agent to treat a separate human or animal (e.g. serum collected from soldiers vaccinated against Anthrax for treatment of a civilian).

In a particular embodiment, the sample is transported to the second location for non-regulated or regulated cGxP (e.g., cGAP for current Good Agricultural Practice, cGLP for current Good Laboratory Practice, cGMP for current Good Manufacturing Practice, and cGTP for current Good Tissue Practice (see FDA guidance)). For example, in a particular embodiment, a tissue sample is transported to the second location for cGTP processing and construct development.

In a fifth aspect, the present invention is a method of deploying a therapeutic agent (e.g., graft, construct or product) to a second or third location for therapeutic use using the deployment kit disclosed herein. In a particular embodiment, the therapeutic agent is suitable for use in restoring skin damaged by disease or injury. In a particular embodiment, the therapeutic agent is suitable for use in restoring bone damaged by disease or injury. In a particular embodiment, the therapeutic agent is an immunotherapy. In a particular embodiment, the therapeutic agent is a vaccine. For example, in a particular embodiment the vaccine is created from appropriately and efficiently harvested, stored, and transported tissue from a patient (e.g a tumor biopsy) using the harvest kit in order to deliver a patient-specific, tumor-specific vaccine at the second location, and then returned using the deployment kit to maintain stable environmental and handling conditions. In a particular embodiment, the therapeutic agent created at the second location is the processed serum collected from a human or animal that has immunity against a disease or infectious agent to treat a separate human or animal (e.g. serum collected from soldiers vaccinated against Anthrax for treatment of a civilian).

In exemplary embodiments, the therapeutic agent is a fully autologous cutaneous construct. In exemplary embodiments, the therapeutic agent is a fully autologous osseous construct. In exemplary embodiments, the therapeutic agent is a fully autologous cartilagenous construct. In exemplary embodiments, the therapeutic agent is a fully autologous osteochondral construct. In exemplary embodiments, the therapeutic agent is a fully autologous muscle construct. In exemplary embodiments, the therapeutic agent is a fully autologous central and/or peripheral nervous system construct. In exemplary embodiments, the therapeutic agent is a fully autologous hepatic construct. In exemplary embodiments, the therapeutic agent is a fully autologous urogenital construct. In exemplary embodiments, the therapeutic agent is a fully autologous gastrointestinal construct. In exemplary embodiments, the therapeutic agent is a fully autologous cardiac construct. In exemplary embodiments, the therapeutic agent is a fully autologous pulmonary construct. In exemplary embodiments, the therapeutic agent is a fully autologous renal construct. In exemplary embodiments, the therapeutic agent is a fully autologous pancreatic construct. In exemplary embodiments, the therapeutic agent is a reproductive cell or cells (e.g. egg, ovum, sperm). In exemplary embodiments, the therapeutic agent is a fully autologous dental construct. In exemplary embodiments, the therapeutic agent is a fully autologous mucosal construct (e.g. oral or vaginal mucosa). In exemplary embodiments, the therapeutic agent is a fully autologous salivary gland construct (e.g. submandibular gland, parotid gland, etc.). In exemplary embodiments, the therapeutic agent is a fully autologous ocular construct.

In exemplary embodiments, the therapeutic agent is a fully allogeneic cutaneous construct. In exemplary embodiments, the therapeutic agent is a fully allogeneic osseous construct. In exemplary embodiments, the therapeutic agent is a fully allogeneic cartilagenous construct. In exemplary embodiments, the therapeutic agent is a fully allogeneic osteochondral construct. In exemplary embodiments, the therapeutic agent is a fully allogeneic muscle construct. In exemplary embodiments, the therapeutic agent is a fully allogeneic central and/or peripheral nervous system construct. In exemplary embodiments, the therapeutic agent is a fully allogeneic hepatic construct. In exemplary embodiments, the therapeutic agent is a fully allogeneic urogenital construct. In exemplary embodiments, the therapeutic agent is a fully allogeneic gastrointestinal construct. In exemplary embodiments, the therapeutic agent is a fully allogeneic cardiac construct. In exemplary embodiments, the therapeutic agent is a fully allogeneic pulmonary construct. In exemplary embodiments, the therapeutic agent is a fully allogeneic renal construct. In exemplary embodiments, the therapeutic agent is a fully allogeneic pancreatic construct. In exemplary embodiments, the therapeutic agent is a fully allogeneic dental construct. In exemplary embodiments, the therapeutic agent is a fully allogeneic mucosal construct (e.g. oral or vaginal mucosa). In exemplary embodiments, the therapeutic agent is a fully allogeneic salivary gland construct (e.g. submandibular gland, parotid gland, etc.). In exemplary embodiments, the therapeutic agent is a fully allogeneic ocular construct.

In one embodiment, the deployment kit is received at the second location within 24, 48, 72, or 96 hours. In one embodiment, the deployment kit is received at a third location (e.g. the location for deployment) within 24, 48, 72, or 96 hours from the time of receiving the harvest kit at the second location. In one embodiment, the deployment kit is received at the location for therapeutic agent deployment after 96 hours from the time of receiving the harvest kit at the second location. In one embodiment, at least a portion of the collected material and/or the therapeutic agent is stored at the second location and the stored portion of the collected material or therapeutic agent is received at the location for deployment upon request at any timepoint. A separate portion can also be sent for immediate deployment. For example, in one embodiment, a portion of a fully autologous cutaneous construct created from a harvested skin biopsy can be stored at a second location, and a separate portion of the fully autologous cutaneous construct can be sent for immediate deployment within 24, 48, 72, or 96 hours.

In a particular embodiment, the therapeutic agent is used to treat a recipient within 24 hours of receipt of the deployment kit. Alternatively, the therapeutic agent is refrigerated (1-10° C.) upon receipt for subsequent use (i.e., greater than 24 hours post-receipt). In a particular embodiment, the therapeutic agent can be used at any time following receipt of the deployment kit with complete environmental control and real-time environmental monitoring data transmission ensuring long-term and indefinite therapeutic agent stability.

### BRIEF DESCRIPTION OF DRAWING

In order that the advantages of the invention will be readily understood, a more particular description of the invention briefly described above will be rendered by reference to specific embodiments that are illustrated in the appended drawing. Understanding that this drawing depicts only typical embodiments of the invention and are not therefore to be considered to be limiting of its scope, the invention will be described and explained with additional specificity and detail through the use of the accompanying drawing, in which:

FIG. 1 depicts an exemplary kit disclosed herein.

### DETAILED DESCRIPTION OF THE INVENTION

Disclosed herein are kits and methods that provide samples such as tissue samples or cellular material for experimental, commercial and/or therapeutic protocols. In one embodiment, the kits and methods of the present invention facilitate extraction of skin tissue from a donor which is then used in preparing a graft for skin restoration.

The kits and methods of the present invention can be used in a range of surgical, medical, cosmetic, research, commercial, agricultural, military, and aerospace applications, to restore or repair cells and/or tissue, to treat or eradicate disease, to create new food sources, to grow and engineer agriculture, to weaponize living materials, and to treat disease or injury in humans and animals distant from Earth (e.g. space station, space shuttle, moon, other planet, etc.).

In order to facilitate the understanding of the present invention in reviewing the drawings accompanying the specification, reference numerals are provided below. It is noted that the drawings are exemplary only.

### REFERENCE NUMERALS

10 Kit
12 Box
14 Insulated mailer
16 Temperature tracker
18 Cooling component
20 Biohazard bag
22 Local anesthetic
24 Vasoconstrictor
26 Absorbent pack
28 Sterile gloves
30 Forceps
32 Scissors
34 Needle driver
36 Antimicrobial agent
38 Scalpel
40 Skin marking device
42 Irrigation syringe
44 Wound dressing

### I. Definitions

As used herein, the singular forms “a,” “an,” “or,” and “the” include plural referents unless the context clearly dictates otherwise.

The term “allogenic” as used herein refers to cells or tissue from different subjects of the same species.

The term “autologous” as used herein refers to cells or tissue from the same subject.

The terms “biological sample,” “tissue sample,” or simply “sample” as used herein refers to a collection of cells or tissue obtained from a tissue, tissues, organ, or organs of a subject. In certain embodiments, the biological sample is a skin biopsy sample.

The term “biopsy cavity” as used herein refers to the void in the subject's tissue after a tissue sample is removed.

The term “biopsy device” as used herein refers to any apparatus which can be used to conduct a biopsy procedure. A biopsy device may be a disposable biopsy device or a biopsy device that includes a disposable portion and a non-disposable portion, where the disposable portion comprises a tissue cutting surface.

The term “comprising” as used herein is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The term “donor” as used herein is taken to mean a subject in which a tissue sample is removed or derived. In one embodiment, the donor will be the same subject as the recipient, i.e., the cell or tissue is autologous. In another embodiment, the donor will be a different subject than the recipient, i.e., the cells or tissues are allogenic.

The term “graft” as used herein refers to any free (unattached) cell, tissue, or organ for transplantation.

The term “harvest” is used to refer to obtaining a sample such as obtaining a blood or tissue sample from a subject.

The term “local anesthetic” as used herein refers to a medication that causes reversible absence of sensation (e.g., pain, pressure, proprioception, etc.). It may optionally include a vasoconstrictor.

The term “needle driver” (also referred to as “needle holder”) as used herein refers to a surgical instrument, used by medical personnel to hold a suturing needle for closing wounds during suturing and surgical procedures.

The term “structural and functional characteristics of a native tissue” refers to the anatomical (structural) and physiological (functional) characteristics of an intact (i.e., not damaged, failing or deficient) native tissue in vivo.

The term “subject” as used herein refers to a mammal, including human and non-human animals.

The term “proliferation” as used herein refers to means to growing or multiplying by producing new cells.

The term “recipient” as used herein refers to the subject to which a cell or tissue is administered (e.g., transplanted).

The term “restore” or “restoration” as used herein refers to any qualitative or quantitative improvement in a target such as in a predetermined tissue or and/or site of treatment observed upon implantation of a cells or tissues.

The term “tissue” as used herein refers to biological tissues, generally defined as a collection of interconnected cells that perform a similar function within an organism. The tissue may be, for example, epithelium, connective tissue, adipose tissue, muscle tissue, bony tissue, nervous tissue, or tissue from hollow or solid organs. In certain embodiments, the tissue is skin.

The term “vasoconstrictor” as used herein refers to an agent that contracts the smooth muscle in blood vessels, which causes the vessels to constrict. Representative, non-limiting vasoconstrictors include alpha-adrenoceptor agonists and vasopressin analogs.

The term “xenogeneic” as used herein refers to cells or tissue from a different species.

### II. Kit

In one aspect, the present invention is a harvesting kit for samples such as cell, tissue, blood, blood-derivative, or body fluid samples. Certain components of the harvest kit are used for transporting a sample (e.g., tissue sample) to a second location, e.g., a location where the sample is used for research or further processed to provide a therapeutic agent (e.g., a graft or construct).

In another aspect, the present invention is a deployment kit such as for deployment of a therapeutic agent formed from a cell, tissue, blood, blood-derivative, or body fluid sample (e.g., a graft, construct or other product). The deployment kit is used to transport agents such as a therapeutic agent to a second location, e.g., a location where a therapeutic agent is used to treat a recipient. In a particular embodiment, the therapeutic agent is an autologous cutaneous construct that arrives at the second location as a semi-liquid homologous suspension or a semi-liquid suspension in a sterile and transparent syringe with plunger.

In the case of a cell, tissue, blood, blood-derivative, or body fluid sample, the sample is obtained from a donor. The donor may be any suitable donor. In a particular embodiment, the donor is a human donor. In another embodiment, the donor is the same human subject as the recipient, i.e., the cells, tissue, blood, blood-derivative, or body fluid are autologous. In a further embodiment, the donor is a different human subject than the recipient, i.e., the cells, tissue, blood, blood-derivative, or body fluid are allogeneic. In yet another embodiment, the donor is a different species than the recipient, i.e., the cells, tissue, blood, blood-derivative, or body fluid are xenogeneic.

The kit of the present invention may also include, alternatively or in combination one or more: insulated mailers or environmentally controlled shipment packages, temperature trackers, cooling components, biohazard bags, irrigation syringes, local anesthetics, vasoconstrictors, absorbent packs, sterile gloves, forceps, scissors, needle drivers, antimicrobial agents, biopsy devices, skin marking devices, labels, dressings and/or suture. In certain embodiments, the kit may contain more than one of the referenced components. For example, the kit may contain more than one biopsy device, label, biohazard bag or dressing.

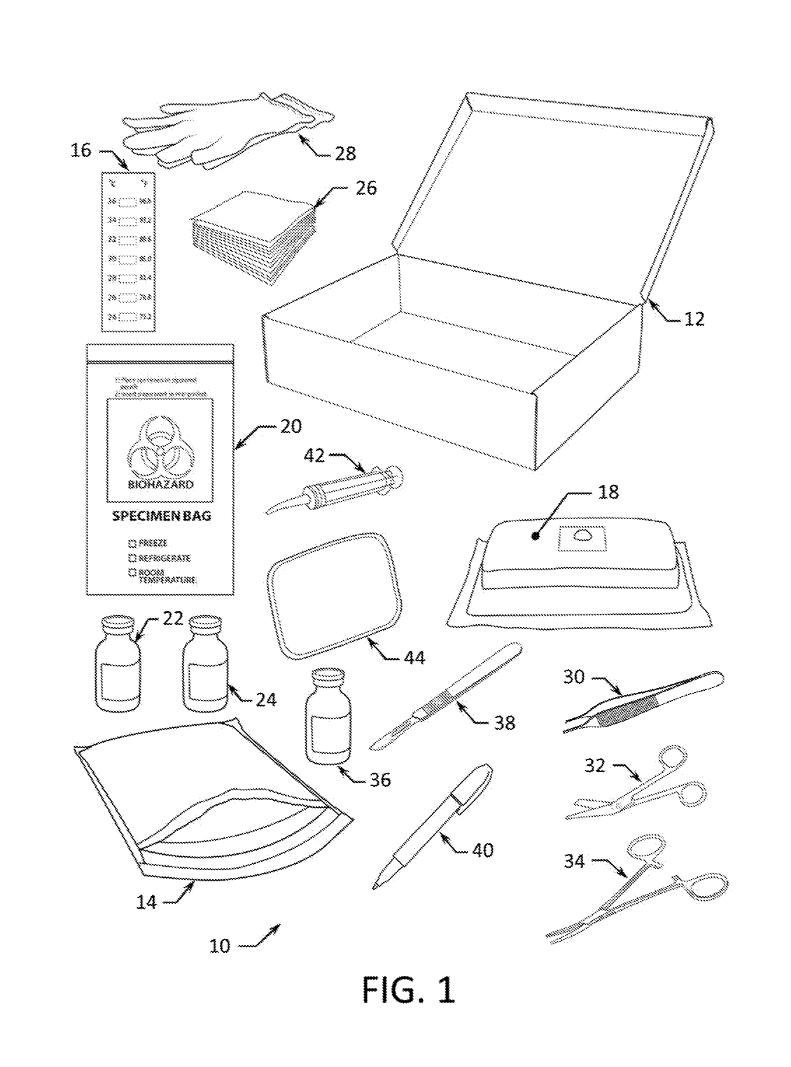

An exemplary kit 10 is shown in FIG. 1. The kit 10 of FIG. 1 includes a box 12 for holding the various components of the kit 10. The kit 10 further includes an insulated mailer 14, a temperature tracker 16, a cooling component 18, a biohazard bag 20, a local anesthetic 22, a vasoconstrictor 24, an absorbent pack 26, sterile gloves 28, forceps 30, scissors 32, a needle driver 34, an antimicrobial agent 36, a scalpel 38, a skin marking device 40, an irrigation syringe 42, and a wound dressing 44.

The syringes in the kit may be made of plastic or the like, and have various volumes. In exemplary embodiments, the syringe is a 60-cc syringe. In another embodiment, the syringe is a 30-cc syringe.

In one embodiment, the kit contains at least one local anesthetic. Local anesthetic anesthetics block the generation and the conduction of nerve impulses. The local anesthetic may be short acting (about 45 minutes to about 90 minutes), intermediate duration (about 90 minutes to about 180 minutes) or long acting (about 4 hours to about 18 hours).

The local anesthetic may be provided in a vial filled with the local anesthetic solution and an appropriately sized syringe (e.g., 10 cc syringe) and needle(s) (e.g., an 18-guage needle for withdrawing the solution and a 30-gauge needle for injecting the solution).

The concentration of the solution may vary. In one embodiment, the concentration of the solution is about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9% or about 0.95% or about 1.0% or greater.

In another embodiment, the concentration of the solution is about 1.0%, about 1.5%, about 2.0%, about 2.5%, about 3.0%, about 3.5%, about 4.0%, about 4.5% or about 5.0% or greater.

Alternatively, local anesthetic may be provided as a topical formulation. In another embodiment, the local anesthetic is provided in the form of a patch.

In certain embodiments, the local anesthetic is prepared for use in iontophoresis.

The local anesthetic may be an amino-amide (“amide-type”) or an amino-ester (“ester-type”) local anesthetics. Representative, non-limiting examples of amide-type local anesthetics include lidocaine, mepivacaine, prilocaine, bupivacaine, etidocaine, ropivacaine and levobupivacaine. Articain is also typically considered an amide-type.

In a particular embodiment, the local anesthetic is lidocaine. In a particular embodiment, the local anesthetic is lidocaine prepared as an injectable solution, e.g., in a vial filled with lidocaine. In a particular embodiment, the injectable solution contains lidocaine in an amount from 1% to 5%, and more particularly, about 1%, about 1.5%, about 2%, about 2.5%, about 3%, about 4% or about 5%. The dose of lidocaine may vary and is to be used at the discretion of the provider.

In exemplary embodiments, the local anesthetic is lidocaine-HCL. In a particular embodiment, the local anesthetic is XYLOCAINE®

In a particular embodiment, the local anesthetic is bupivacaine. In a particular embodiment, the bupivacaine is 0.25% bupivacaine or 0.50% bupivacaine prepared as an injectable solution. The dose may vary and is to be used at the discretion of the provider.

In exemplary embodiments, the local anesthetic is bupivacaine-HCL. In a particular embodiment, the local anesthetic is MARCAINE™.

In one embodiment, the local anesthetic comprises two or more amide-type local anesthetics. In a particular embodiment, the local anesthetic comprises lidocaine and bupivacaine.

Representative, non-limiting examples of ester-type local anesthetics include chloroprocaine, procaine, tetracaine, benzocaine and salts thereof.

The local anesthetic may be combined with a vasoconstrictor, for example epinephrine. Addition of a vasoconstrictor may beneficially decrease in the peak plasma concentration of the local anesthetic agent, increase the duration and the quality of anesthesia, reduce of the minimum concentration of anesthetic needed for nerve block and/or decrease of blood loss during surgical procedures

In exemplary embodiments, the local anesthetic is lidocaine-epinephrine or XYLOCAINE®-epinephrine. In a particular embodiment, the ratio of epinephrine is 1:100,000 or 1:200,000. The dose may vary and is to be used at the discretion of the provider.

In exemplary embodiments, the local anesthetic is bupivacaine-epinephrine or MARCAINE™-epinephrine. In a particular embodiment, the ratio of epinephrine is 1:100,000 or 1:200,000. The dose may vary and is to be used at the discretion of the provider.

Optionally, one or more additives may be included in the local anesthetic prepared for injection. In a particular embodiment, sodium bicarbonate is included in the local anesthetic.

The dressing may be any suitable dressing. In one embodiment, the dressing is impregnated with at least one antimicrobial. The dressing may be, for example, a gauze or bandage, naturally-occurring, synthetic, or semi-synthetic compound or composition or mixture thereof. In a particular embodiment, the antimicrobial is selected from the group consisting of iodine, silver, honey or methyl blue. In a particular embodiment, the dressing is occlusive, nonadherent, and nonporous. In an exemplary embodiment, the antimicrobial dressing is an ioban dressing. In an exemplary embodiment, the dressing is a silicone dressing.

The biopsy device may be any suitable biopsy device, based on the method utilized. In one embodiment, the biopsy device is selected from the group consisting of a scalpel blade, a needle biopsy device, hookwire biopsy device, photonic needle, clamp, forceps, micro-scissors, punch biopsy device, core biopsy device, razor, shave biopsy device, a cutaneous needle biopsy device. In one embodiment, the biopsy device is a disposable biopsy device. In some embodiments, more than one disposable biopsy device is provided, e.g., one, two, three, four or more disposal biopsy devices. In another embodiment, the kit includes a disposable attachment for a non-disposable biopsy device. In some embodiments, the biopsy device includes blood collection needles, blood collection tubes, a tourniquet, and a syringe.

In an exemplary embodiment, the biopsy device is a scalpel. The skin marking device may be any suitable skin marking device. In one embodiment, the skin marking device is a surgical marker. In another embodiment, the skin marketing device is a syringe which dispenses a colorant to give a visual indication on the surface of the skin of the point at which an injection has or will be given. In a further embodiment, the skin marking device is a device which has patterning elements for impressing a temporary mark on the surface of the skin.

The label may be any suitable label. In certain embodiments, the kit contains multiple labels. The labels may be marked, for example, with a patient identification number (PIN). In certain embodiments, the labels ensure sample and therapeutic agent traceability to ensure donor and recipient identification. In certain embodiments, the labels may be marked with de-identified donor and recipient tracking information. In certain embodiments, the de-identified donor and recipient tracking information may be marked on the labels with other transport tracking labels and numbers in order to geolocate donor and recipient specific samples and therapeutic agents in real-time.

Optionally, one or more additional components may be included in the kit.

In one embodiment, the kit further comprises a skin preparation agent. In one embodiment, the skin preparation is an antiseptic selected from isopropyl alcohol, povidone-iodine solution, or chlorhexidine.

In another embodiment, the kit further comprises a sterile container for storing the tissue sample. The container may include a label and identifying information such as a bar code.

In another embodiment, the kit further comprises forceps.

In a further embodiment, the kit further comprises a hemostatic agent.

In certain embodiments, the kit is expressly adapted to enable a harvested tissue sample to arrive at a remote location in a minimally altered or substantially pristine state (e.g. minimally altered by the environmental effects that could otherwise alter the tissue sample). In certain embodiments, the kit further includes means for remote monitoring and communication (such as an audio/video transponder means for immediate real-time audio and video communication between a kit user and product support personnel) with the kit and more especially real-time monitoring of various trackable data of the kit. For instance, the kit may include a GPS transponder chip to enable the real-time location tracking of the kit. Further, sensors and means to transmit sensed data such as kit temperature, kit temperature changes, kit loading (e.g. shock, vibration, and impact loading (e.g. from the kit being dropped), kit spatial positioning (e.g. kit is positioned sideways or upside down), and atmospheric sensing (e.g. oxygen, nitrogen, and other gas levels, moisture/desiccation levels, barometric pressure, altitude). Further the kit may include microbial monitoring and microbial control means. The kit may also include personal protective equipment such as a lab coat, safety glasses, and a mask. In certain embodiments, the kit includes physical and/or digital security features (including for instance biometric locks), traceability features (including for instance biometric traceability), tamper prevention features (for instance a mechanical key or combination lock), and tamper evident features (such as tamper evident security tape). In some embodiments the kit includes organic samples. In other embodiments, the kit includes inorganic samples. In still further embodiments, the kit contains no samples (but is adapted to be used for collecting, storing, transporting, or deploying a sample).

### III. Methods of Use

In one aspect, the present invention is a method of harvesting samples such as harvesting cell, tissue, blood, blood-derivative, or body fluid samples using the disclosed harvesting kit.

The cells or tissues may be harvested by any suitable method. In one embodiment, the cells or tissue are harvested by biopsy. The biopsy method may be any suitable method, based on the desired size and/or components of the sample. In one embodiment, the biopsy method is an open biopsy. In another embodiment, the biopsy method is a percutaneous biopsy.

In one embodiment, the tissue is skin. In an exemplary embodiment, the method comprises (i) cleaning the skin; (ii) injecting an effective amount of local anesthetic (e.g., lidocaine-epinephrine) into a target site to produce an insensate area of the skin; (ii) biopsying the skin within the numb area using a scalpel or skin punch; (iv) excising the biological sample to provide a tissue sample and a tissue cavity; (iv) placing the tissue sample in a specimen container; (v) closing the tissue cavity with suture to provide a biopsy site; and (vi) dressing the biopsy site with appropriate dressings.

In one embodiment, the method further comprising placing the specimen container within the biohazard bag then placing the biohazard bag in the insulated mailer, together with the cooling component.

In one embodiment, the skin biopsy is partial. In another embodiment, the skin biopsy is full thickness.

In an exemplary embodiment, skin biopsy is a full-thickness skin biopsy with a minimum surface area of 2 cm². In an exemplary embodiment, skin biopsy is a full-thickness skin biopsy with a minimum surface area of 1 cm².

In one embodiment, a full-thickness skin excisional biopsy is harvested in a manner that is consistent with current clinical standard of care utilizing the materials provided in the kit. In another embodiment, the sample of skin is excised by a biopsy technique selected from punch or excisional techniques.

Excisional Biopsy.

In an exemplary embodiment, the biopsy technique is excisional. In one embodiment, the excisional biopsy technique is elliptical excision. In one embodiment, the excisional biopsy is accomplished using a scalpel, forceps, and/or scissors. The excisional biopsy captures the full-thickness of the skin including the epidermis, dermis, and partial to complete layers of the hypodermal fat.

In an exemplary embodiment, the skin biopsy is an excisional, full-thickness skin biopsy with a minimum surface area of 1 cm². In an exemplary embodiment, the skin biopsy is an excisional, full-thickness skin biopsy with a minimum surface area of 1 cm². In a particular embodiment, the skin biopsy is an excisional, full-thickness skin biopsy with a surface area of about 1 cm², about 2 cm², about 3 cm², about 4 cm², about 5 cm², or greater than about 5 cm².

Punch Biopsy.

In an exemplary embodiment, the biopsy technique is punch biopsy. A punch biopsy will provide a full-thickness biopsy of skin ranging in diameter from 2- to 10-mm, based on the size of the punch biopsy instrument. The punch biopsy yields a cylindrical core of tissue that includes the epidermis, the dermis and the subcutaneous tissue. In a particular embodiment, the punch biopsy provides a full-thickness cylindrical core of skin about 2, about 3, about 4, about 5, about 6, about 7, about 8, about 9 or about 10 mm in diameter.

Excisional and/or punch biopsy tissue cavities can be closed, if required at the discretion of the provider, with suture using a needle driver, forceps, and/or scissors contained within the harvesting kit.

In another embodiment, the biopsy is a core needle biopsy that removes cells as well as a small amount of the surrounding tissue.

Advantageously, utilizing the kit increases the efficiency of obtaining a sample such as a tissue specimen and/or transporting it to the second location. In certain embodiments, utilizing the kit decreases the risk of contamination of the sample.

In another aspect, the present invention is a method of preserving and optionally transporting the sample such as cells or tissues to a second location. The kits disclosed herein advantageously preserves viability of the transported sample. As use used herein, the term “viability” means that after storage and/or transport, the sample functions for its intended purpose such as in the case of a tissue sample the cells are alive and capable of the same functions in existence prior to storage.

In some embodiments, the cells retain greater than 70% of the viability of the initial population after storage and/or transport.

In one embodiment, the harvest kit is received at the second location for use within 24, 48 or 72 hours of the harvest.

In one embodiment, a tissue sample is placed into a sterile container, which optionally contains transport medium or crystalloid solution, and optionally antibiotic, and then placed within two biohazard bags, one with an absorbent pad, for shipping, optionally together with the cooling component (e.g., freezer pack, dry ice, wet ice, or liquid nitrogen), to maintain the contents at a suitable temperature. In exemplary embodiments, the suitable temperature is above freezing. In one embodiment, the suitable temperature is about 4° C. The temperature tracker may be used to ensure that the temperature remains within a suitable range. The cooling system may be designated for overnight shipping, for both the transport of a tissue sample after harvest and transport of a tissue sample derived graft for deployment, as part of the deployment box, e.g., FedEx (or like carrier) priority overnight (before 10 am the next day) or first overnight (before 8 am the next day).

The sample such as a tissue sample or cells are suitable for use in research and certain protocols such as therapeutic protocols.

In one embodiment, the method further comprises processing a tissue sample obtained to provide a therapeutic agent, e.g., a graft, construct or other product. The therapeutic agent may be autologous, allogenic, or xenogeneic.

The methods of processing a tissue sample include any suitable method. In a particular embodiment, the methods of processing are cGTP. In a particular embodiment, the methods of processing are cGMP. In a particular embodiment, the methods of processing are cGLP.

In one embodiment, a tissue sample is tested pre-processing and shown to be free of bacterial and fungal pathogens. Certain non-pathogenic skin bacteria may be present.

In one embodiment, the therapeutic agent is tested post-processing and shown to be free of bacterial and fungal pathogens. Certain non-pathogenic skin bacteria may be present.

In a particular embodiment, a tissue sample is completely processed within 24 or 48 hours of receiving the harvest kit.

In one aspect, the present invention is a method for deploying a therapeutic agent formed from a harvested tissue sample using the deployment kit.

The deployment kit contains at least one therapeutic agent. The agent may be an autologous, allogenic or xenogeneic therapeutic agent. In a particular embodiment, the therapeutic agent is an autologous graft, construct or other product. In another particular embodiment, the therapeutic agent is an allogenic graft, construct or other product.

In a particular embodiment, the therapeutic agent is a fully autologous, homogenous cutaneous construct for reconstruction and/or regeneration of skin. It may be used as an adjunct and/or in place of split-thickness skin grafting, full-thickness grafting, temporizing skin coverage and/or skin substitute products.

The deployment kit may contain a cooling component and at least one biohazard bag. In a particular embodiment, the deployment kit contains a cooling component and with (3) additional sequential barrier bags.

In a particular embodiment, the construct is applied or deployed into a target site (e.g., wound bed) that has been properly treated (such as by a medical professional). The treatment may include debriding, excision, sterile preparation and/or cleaning.

In one embodiment, the deployment kit is received at the second location for therapeutic use within 48 hours or 72 hours from the time the biopsy was obtained.

Disclosed herein is a kit for use in harvesting and deploying a tissue sample comprising: an insulated mailer; a temperature tracker; a cooling component; a sterile container; and at least one of a local anesthetic, a vasoconstrictor, an absorbent pack, sterile gloves, a needle driver, an antimicrobial agent, a biopsy device, a skin marking device, and a biohazard bag.

In embodiments, the cooling component is at least one of a freezer pack, dry ice, wet ice, and liquid nitrogen. In embodiments, the kit further comprises an irrigation syringe. In embodiments, the irrigation syringe is a 30 cc irrigation syringe. In embodiments, the kit further comprises a wound dressing. In embodiments, the wound dressing is silicone. In embodiments, the wound dressing is antimicrobial. In embodiments, the local anesthetic is lidocaine and the vasoconstrictor is epinephrine. In embodiments, the antimicrobial agent is gentamicin. In embodiments, the biopsy device is at least one of a scalpel, a needle biopsy device, a hookwire biopsy device, a photonic needle, a clamp, forceps, micro-scissors, a punch biopsy device, a core biopsy device, a razor, a shave biopsy device, and a cutaneous needle biopsy device. In embodiments, the skin marking device is at least one of a surgical marker, a colorant dispensing syringe, and a temporary skin pattern impressing device. In embodiments, the biohazard bag is a UN3373 biohazard bag. In embodiments, the kit includes a plurality of the local anesthetic, the vasoconstrictor, the absorbent pack, sterile gloves, the needle driver, the antimicrobial agent, the biopsy device, the skin marking device, and the biohazard bag. In embodiments, the sterile container is a 50 cc conical tube. In embodiments, the kit further comprises a skin preparation agent. In embodiments, the skin preparation agent is povidone iodine. In embodiments, the kit further comprises a geographical locating device. In embodiments, the geographical locating device is a GPS transponder chip. In embodiments, the kit further comprises an environmental monitoring device with or without remote and distant data transmission and storage capabilities. In embodiments, the environmental monitoring device with or without remote and distant data transmission and storage capabilities is at least one of an analog or digital temperature monitor, a pressure sensor, an accelerometer, a particle counter, and an air composition monitor. In embodiments, the kit further comprises antimicrobial monitoring. In embodiments, the kit further comprises a security feature. In embodiments, the security feature is at least one of a physical lock, a digital code entry screen, a key card access point, a biometric scanner, biometric data storage and tracing, a tamper indicator, and a leak detector. In embodiments, the kit further comprises a mechanism to be connected to transportation vehicles. In embodiments, the mechanism to be connected to transportation vehicles is at least one of a physical mechanism to be carried by an unmanned aerial vehicle, a mechanism to attach to other similar kits for grouped transport and movement, a mechanism for a group of connected kits to be carried by an unmanned aerial vehicle, a mechanism to attach to the internal or external surface of motorized or non-motorized transportation devices, a robotic self-driving chassis, a mechanism to attach to the internal or external surface of a self-driving vehicle, and a mechanism to attach to the internal or external surface of an object intended to travel beyond Earth's atmosphere.

Also disclosed herein is a method of using a kit to harvest a skin tissue sample from a subject, comprising (i) cleaning the skin; (ii) injecting an effective amount of the local anesthetic to produce a insensate skin area; (iii) biopsying the skin at a target site within the insensate skin area using the biopsy device; (iv) excising the biopsy to provide a tissue sample and a tissue cavity; (v) placing the tissue sample in a specimen container; (vi) closing the tissue cavity with suture to provide a biopsy site; and (vii) dressing the biopsy site with appropriate dressings, wherein the kit comprises: (i) an insulated mailer; (ii) a temperature tracker, (iii) a cooling component, (iv) a biohazard bag, (v) a local anesthetic, (vi) a vasoconstrictor, (vii) an absorbent pack, (viii) sterile gloves, (ix) forceps, (x) scissors, (xi) a needle driver, (xii) an antimicrobial agent, (xiii) a biopsy device, and (xiv) a skin marking device.

In embodiments, the method further comprises placing the tissue sample in a biohazard bag. In embodiments, the biohazard bag further comprises a cooling component. In embodiments, the biopsy device is at least one of a scalpel, a needle biopsy device, a hookwire biopsy device, a photonic needle, a clamp, forceps, micro-scissors, a punch biopsy device, a core biopsy device, a razor, a shave biopsy device, and a cutaneous needle biopsy device. In embodiments, the tissue sample is a full-thickness sample of skin.

Further disclosed herein is a method of using a kit to preserve and transport a tissue sample to a second location comprising adding the tissue sample to a specimen container; enclosing the specimen container within a first biohazard bag: enclosing the first biohazard bag containing the specimen container within a second biohazard bag containing the absorbent pad; enclosing the first and second biohazard bags within an insulated mailer, wherein the insulated mailer comprises a cooling component and a temperature tracker; and transporting the insulated mailer to the second location, wherein the kit comprises: (i) an insulated mailer; (ii) a temperature tracker, (iii) a cooling component, (iv) a biohazard bag, (v) a local anesthetic, (vi) a vasoconstrictor, (vii) an absorbent pack, (viii) sterile gloves, (ix) forceps, (x) scissors, (xi) a needle driver, (xii) an antimicrobial agent, (xiii) a biopsy device, and (xiv) a skin marking device.

In embodiments, the tissue sample arrives at the second location in less than 24 hours after the biopsy. In embodiments, the tissue sample is maintained at about 4° C. during transport.

In embodiments, the viability of the tissue sample is maintained during transport.

Disclosed herein is a kit for use in harvesting and deploying a tissue sample comprising: (i) an insulated mailer; (ii) a temperature tracker, (iii) a cooling component, (iv) a biohazard bag, (v) a local anesthetic, (vi) a vasoconstrictor, (vii) an absorbent pack, (viii) sterile gloves, (ix) forceps, (x) scissors, (xi) a needle driver, (xii) an antimicrobial agent, (xiii) a biopsy device, and (xiv) a skin marking device.

In embodiments, the cooling component is at least one of a freezer pack, dry ice, wet ice, and liquid nitrogen. In embodiments, the biopsy device is at least one of a scalpel, a needle biopsy device, a hookwire biopsy device, a photonic needle, a clamp, forceps, micro-scissors, a punch biopsy device, a core biopsy device, a razor, a shave biopsy device, and a cutaneous needle biopsy device. In embodiments, the skin marking device is at least one of a surgical marker, a colorant dispensing syringe, and a temporary skin pattern impressing device. In embodiments, kit includes at least one of an irrigation syringe, a wound dressing, and a skin preparation agent. In embodiments, the local anesthetic is lidocaine, the vasoconstrictor is epinephrine, and the antimicrobial agent is povidone iodine.

Disclosed herein is a method of harvesting a skin tissue sample, comprising providing a kit having an insulated mailer; a temperature tracker; a cooling component; a biohazard bag; a biopsy device; a local anesthetic; and at least one of a vasoconstrictor, an absorbent pack, sterile gloves, a needle driver, an antimicrobial agent, and a skin marking device; cleaning the skin; injecting an effective amount of the local anesthetic to produce a insensate skin area; biopsying the skin at a target site within the insensate skin area using the biopsy device; excising the biopsy to provide a tissue sample; and placing the tissue sample in the insulated mailer.

In embodiments, the cooling component is at least one of a freezer pack, dry ice, wet ice, and liquid nitrogen. In embodiments, the biopsy device is at least one of a scalpel, a needle biopsy device, a hookwire biopsy device, a photonic needle, a clamp, forceps, micro-scissors, a punch biopsy device, a core biopsy device, a razor, a shave biopsy device, and a cutaneous needle biopsy device. In embodiments, the skin marking device is at least one of a surgical marker, a colorant dispensing syringe, and a temporary skin pattern impressing device. In embodiments, kit includes at least one of an irrigation syringe, a wound dressing, and a skin preparation agent. In embodiments, the local anesthetic is lidocaine, the vasoconstrictor is epinephrine, and the skin preparation agent is povidone iodine.

Disclosed herein is a method of using a tissue sample comprising providing a kit having an insulated mailer that includes a temperature tracker; a cooling component; a specimen container; a biohazard bag; a biopsy device; a local anesthetic; and at least one of a vasoconstrictor, an absorbent pack, sterile gloves, a needle driver, an antimicrobial agent, and a skin marking device; placing a tissue sample in a specimen container; enclosing the specimen container and the absorbent pack within a biohazard bag; placing the biohazard bag within the insulated mailer; and transporting the insulated mailer to a second location.

In embodiments, the cooling component is at least one of a freezer pack, dry ice, wet ice, and liquid nitrogen. In embodiments, the biopsy device is at least one of a scalpel, a needle biopsy device, a hookwire biopsy device, a photonic needle, a clamp, forceps, micro-scissors, a punch biopsy device, a core biopsy device, a razor, a shave biopsy device, and a cutaneous needle biopsy device. In embodiments, the skin marking device is at least one of a surgical marker, a colorant dispensing syringe, and a temporary skin pattern impressing device. In embodiments, the kit includes at least one of an irrigation syringe, a wound dressing, and a skin preparation agent. In embodiments, the local anesthetic is lidocaine, the vasoconstrictor is epinephrine, and the antimicrobial agent is povidone iodine. In embodiments, the tissue sample arrives at the second location in less than 72 hours after the tissue sample is placed in the specimen container. In embodiments, the tissue sample is maintained at a temperature in the range of 0° C. to 10° C. during said transporting.

### EXAMPLES

### Example 1: Use of the Harvesting Kit

1. Prepare a full-thickness skin excisional biopsy site in a manner that is consistent with current clinical standard of care utilizing the materials provided in harvest kit and in particular, as described in Example 2.
2. Once harvested, place the excisional skin biopsy in the sterile container and add transport solution.
3. Close the sterile container tightly and add to the biohazard bag.
4. Place a label on the biohazard bag in order to correlate with the patient identification number (PIN).
5. Place the labeled biohazard bag containing the tissue specimen into a second biohazard bag containing an absorbent pad.
6. Place the packaged specimen into the included NanoCool® insulated container and activate the NanoCool® cooling system by pressing the button on the underside of the NanoCool® lid. The package should become cool within 1 minute.
7. Activate the temperature tracker strip stuck to the bottom of the NanoCool® lid.
8. Close the NanoCool® box and ensure that there is a pre-filled shipping label already in place.
9. Secure the lid of the box with a tamperproof sealing label and contact the FedEx® carrier number on shipping label for same day pick up.

### Example 2: Harvesting the Tissue Sample

Prepare the biopsy site using surgical betadine prep and drape in standard fashion to ensure sterility.
Inject local anesthetic in standard clinical fashion.
Sharply excise a full-thickness specimen including epidermis to fat and do not defat the specimen.

1. A kit for use in harvesting and deploying a tissue sample comprising: an insulated mailer; a temperature tracker; a cooling component; a sterile container; and at least one of a local anesthetic, a vasoconstrictor, an absorbent pack, sterile gloves, a needle driver, an antimicrobial agent, a biopsy device, a skin marking device, and a biohazard bag.

2. The kit of claim 1, wherein the cooling component is at least one of a freezer pack, dry ice, wet ice, and liquid nitrogen.

3. The kit of claim 1, further comprising an irrigation syringe.

4. The kit of claim 1, further comprising a wound dressing.

5. The kit of claim 4, wherein the wound dressing is antimicrobial.

6. The kit of claim 1, wherein the local anesthetic is lidocaine and the vasoconstrictor is epinephrine.

7. The kit of claim 1, wherein the antimicrobial agent is gentamicin.

8. The kit of claim 1, wherein the biopsy device is at least one of a scalpel, a needle biopsy device, a hookwire biopsy device, a photonic needle, a clamp, forceps, micro-scissors, a punch biopsy device, a core biopsy device, a razor, a shave biopsy device, and a cutaneous needle biopsy device.

9. The kit of claim 1, wherein the skin marking device is at least one of a surgical marker, a colorant dispensing syringe, and a temporary skin pattern impressing device.

10. The kit of claim 1, wherein said kit includes a plurality of the local anesthetic, the vasoconstrictor, the absorbent pack, sterile gloves, the needle driver, the antimicrobial agent, the biopsy device, the skin marking device, and the biohazard bag.

11. The kit of claim 1, further comprising a skin preparation agent.

12. The kit of claim 1, further comprising a geographical locating device.

13. The kit of claim 1, further comprising an environmental monitoring device with or without remote and distant data transmission and storage capabilities.

14. The kit of claim 1, further comprising antimicrobial monitoring.

15. The kit of claim 1, further comprising a security feature.

16. A method of harvesting a skin tissue sample, comprising providing a kit having an insulated mailer; a temperature tracker; a cooling component; a biohazard bag; a biopsy device; a local anesthetic; and at least one of a vasoconstrictor, an absorbent pack, sterile gloves, a needle driver, an antimicrobial agent, and a skin marking device; cleaning the skin; injecting an effective amount of the local anesthetic to produce a insensate skin area; biopsying the skin at a target site within the insensate skin area using the biopsy device; excising the biopsy to provide a tissue sample; and placing the tissue sample in the insulated mailer.

17. The method of claim 16, wherein the cooling component is at least one of a freezer pack, dry ice, wet ice, and liquid nitrogen.

18. The method of claim 16, wherein the biopsy device is at least one of a scalpel, a needle biopsy device, a hookwire biopsy device, a photonic needle, a clamp, forceps, micro-scissors, a punch biopsy device, a core biopsy device, a razor, a shave biopsy device, and a cutaneous needle biopsy device.

19. The method of claim 16, wherein the skin marking device is at least one of a surgical marker, a colorant dispensing syringe, and a temporary skin pattern impressing device.

20. The method of claim 16, wherein said kit includes at least one of an irrigation syringe, a wound dressing, and a skin preparation agent.
